(19) United States
(12) Patent Application Publication
(10) Pub. No.: US 2025/0043318 A1
(43) Pub. Date: Feb. 6, 2025

(54) PROCESS FOR HYDRATING YEASTS IN DEHYDRATED FORM

(71) Applicant: VERSALIS S.P.A., SAN DONATO MILANESE (MI) (IT)

(72) Inventors: Valentina RODIGHIERO, Novara (NO) (IT); Elena GASTALDO, Rivalta Scrivia (AL) (IT); Piero OTTONELLO, Rivalta Scrivia (AL) (IT); Alessandro DEL SEPPIA, Mantova (MN) (IT); Tommaso PRANDO, Novara (NO) (IT); Alessandra FRATTINI, Rivalta Scrivia (AL) (IT)

(21) Appl. No.: 18/718,340

(22) PCT Filed: Dec. 6, 2022
(86) PCT No.: PCT/IB2022/061802

§ 371 Date: Jun. 10, 2024

(30) Foreign Application Priority Data
Dec. 10, 2021 (IT) 102021000031070

(51) Int. Cl. C12P 7/10; C12N 1/18; C12N 1/22

(52) U.S. Cl. CPC C12P 7/10; C12N 1/18; C12N 1/22

(57) ABSTRACT

A process for hydrating a yeast in dehydrated form including the following steps
preparing a hydration medium having yeast extract in aqueous solution, at a concentration between 1 g/L and 50 g/L;
adding a yeast in dehydrated form to the hydration medium at a concentration between 10 g/L and 200 g/L;
the process being carried out at a temperature between 25° C. and 40° C., for a time between 30 minutes and 8 hours.

The rehydrated yeast thus obtained may be advantageously used in fermentation processes that allow the production of biofuels usable in diesel or aviation engines such as, ethanol, linear or branched alcohols with different molecular weight, or biochemicals such as, for example, ethanol, glycerol, acetic acid esters (for example, ethyl acetate), arabinitol, lactic acid, succinic acid, microbial oils (bio-oils), or propagated yeast, in particular in fermentation processes carried out in the presence of hydrolyzates deriving from lignocellulosic biomass.

### CROSS-REFERENCE TO RELATED APPLICATIONS

This application is a 35 U.S.C. § 371 National Stage patent application of PCT/IB2022/061802, filed on Jun. 12, 2022, which claims the benefit of Italian patent application 102021000031070, filed on Oct. 12, 2021, the disclosures of which are incorporated herein by reference in their entirety.

### TECHNICAL FIELD

The present disclosure relates to a process for hydrating yeasts in dehydrated form.

More particularly, the present disclosure relates to a process for hydrating a yeast in dehydrated form, comprising: preparing a hydration medium comprising yeast extract in aqueous solution, at a specific concentration: adding a yeast in dehydrated form to said hydration medium at a specific concentration; said process being carried out under specific conditions of temperature and time.

The rehydrated yeast thus obtained can be advantageously used in fermentation processes that allow the production of biofuels usable in diesel or aviation engines such as, for example, ethanol, linear or branched alcohols with different molecular weight, or biochemicals such as, for example, ethanol, glycerol, acetic acid esters (for example, ethyl acetate), arabinitol, lactic acid, succinic acid, microbial oils (bio-oils), or propagated yeast, in particular in fermentation processes carried out in the presence of hydrolyzates deriving from lignocellulosic biomass.

Consequently, the present disclosure also relates to a fermentation process for the production of biofuels or biochemicals comprising at least one yeast deriving from the aforementioned hydration process.

### BACKGROUND

The implementation of processes for the production of biofuels or biochemicals from renewable sources is of great interest for the development of “green” processes, in particular if said processes exploit renewable sources that are not in competition with the food sector such as, for example, lignocellulosic biomass.

One of the major critical issues affecting the development of these “green” processes at an industrial level is the fact that the hydrolyzates deriving from the hydrolysis of lignocellulosic biomasses have a high content of toxic compounds such as, for example, furfural (F), 5-hydroxymethylfurfural (HMF), organic acids (for example, formic acid, acetic acid), phenolic compounds deriving from lignin (for example, vanillin, syringaldehyde), which can act as growth inhibitors of microorganisms usually used in fermentation such as, for example, yeasts, and cause a long adaptation phase (lag phase), which is necessary for microorganisms to adapt to unfavorable growth conditions.

Due to said long adaptation phase (lag phase), a higher consumption of microorganisms (for example, yeast) may be necessary, or the use of complex setup implants due to the fact that it may be necessary to carry out the process in fed-batch mode, or longer propagation times, or the use of multiple propagators, in order to respect the timing of fermentation. It may also be necessary to dilute or detoxify the streams entering the fermentation plant. Furthermore, the presence of a long adaptation phase (lag phase) can expose the culture of the microorganism to a risk of contamination.

The adaptation phase (lag phase) is defined as a temporary period of non-replication of a microbial population, wherein the cells of the microorganism adapt to the new environment before starting the actual growth: during this phase, the cells of the microorganism are not dormant but metabolically active, even if no cell duplication is observed. Said adaptation phase (lag phase) can include, for example, the reparation of the damage suffered by the macromolecules during the conservation of the microorganism and the synthesis of cellular components necessary for its growth, the exploitation of the nutrients present in the available culture medium and, possibly, protection from the existing toxic compounds. Many factors can influence the duration of the adaptation phase (lag phase), including the extent of inoculation of the microorganism, the physiological history of the microorganism's cells and the chemical-physical characteristics of the environment of origin of the microorganism and of the new culture medium.

For example, in the case of using hydrolyzates deriving from lignocellulosic biomass in the fermentation, if the concentration of inhibitors, individually or synergistically, approaches the tolerance threshold of the microorganism (for example, yeast), a long adaptation phase (lag phase) at the beginning of the propagation phase of the microorganism can be required, linked to the need to adapt to the toxic conditions of the new culture medium and to synthesize the enzymes essential for the detoxification. Said adaptation phase (lag phase) is even more impactful if a dehydrated yeast is used, which is in a condition of greater sensitivity.

A particularly long adaptation phase (lag phase) (a few hours) can significantly impact the overall duration of propagation of the microorganism, which is itself a short-lived step (maximum 30 hours): this can take, depending on the plant requirements, as reported above, the presence of a greater number of propagators or a higher yeast consumption to respect the fermentation times. Furthermore, as reported above, the permanence in the culture medium in the optimal conditions of propagation of a microorganism not in active growth, exposes the culture to the risk of biological contamination, especially on an industrial scale: this contamination can also occur when the adaptation phase (lag phase) occurs in the fermentation step, in the case of direct inoculation (direct pitching).

On a laboratory scale, the main revitalization strategy of a dehydrated (or frozen) microorganism consists in the use of a pre-inoculum: for this purpose, the microorganism is inoculated at a low concentration in a medium rich in nutrients such as, for example, sources of organic nitrogen, vitamins, microelements, carbon sources, incubated under suitable conditions and left to grow up to a concentration sufficient to inoculate the actual propagation (8 hours-24 hours), which is carried out in the culture medium of interest, or the fermentation. However, in the production of biofuels or biochemicals on an industrial scale, the aforesaid method is hardly practicable, because it requires large volumes of culture medium, significant quantities of high-cost nutrients (for example, peptone, yeast extract) and controlled growth conditions, with significant impacts on process costs.

Generally, the rehydration of yeasts in dehydrated form on an industrial scale is carried out according to the instructions of the yeast supplier, which involves hydration at high concentration in small volumes of water (not demineralized) or physiological solution (for example, NaCl 0.9% by weight), at the optimum temperature for the yeast in question (20° C.-45° C.), for short periods of time (<1 hour): the yeast thus rehydrated can be inoculated directly during fermentation (direct pitching) or propagated through a propagation phase.

To reduce the adaptation phase (lag phase) due to the presence of inhibitors, alternative strategies can be used on an industrial scale such as, for example, the preliminary detoxification of the hydrolyzate deriving from lignocellulosic biomass with various techniques, or the use of alternative propagation processes with dilution or partial replacement of the hydrolyzate with other carbon sources: however, both solutions increase the complexity of the process and can, in some cases, impact on the final concentration of the yeast.

In the literature, there are numerous strategies to improve the performance of yeasts, or their propagation, even on second-generation sugars such as, for example, sugars contained in hydrolyzates deriving from lignocellulosic biomass, which also include advanced hydration techniques.

For example, US patent U.S. Pat. No. 9,340,767 relates to a method of propagating one or more organisms in a culture medium that uses a carbon source that includes xylose [for example, xylose syrup resulting from the pretreatment of lignocellulosic raw material (feedstock)] and/or a source of nutrients that includes a distillate (stillage component) (for example, a distillate resulting from the fermentation process from corn to ethanol). Said organisms include those capable of converting one or more monosaccharide sugars into alcohol by fermentation, for example, yeasts. Preferably, a genetically modified strain of Saccharomyces cerevisiae is used. The above method is said to be able to improve propagation, as xylose does not trigger the so-called “Crabtree effect”, which shifts the metabolism towards the production of ethanol even in the presence of oxygen. At the end of the propagation, the yeast is inoculated to fermentation.

US patent application US 2015/0252319 relates to a process for the aerobic propagation of yeast wherein the yeast is grown in a reactor, comprising the following steps: a) feeding the reactor with a carbon source and an initial yeast population: b) optionally growing the initial yeast population in the reactor in fed-batch mode: c) measuring the pH in the reactor: d) adding the hydrolyzate deriving from lignocellulosic biomass into the reactor in fed-batch mode at a rate such as to fix the pH in the reactor at a predetermined value; and e) after sufficient propagation, isolating the yeast from the reactor. Preferably, a genetically modified strain of Saccharomyces cerevisiae is used and the hydrolyzate deriving from lignocellulosic biomass is diluted with water.

The international patent application WO2017/144389 relates to a propagation process of a yeast capable of fermenting the glucose and xylose contained in a hydrolyzate deriving from lignocellulosic biomass, said process comprising the propagation of yeast for at least two propagation cycles. The first propagation cycle comprises the steps of: contacting the yeast at an initial yeast density with a first cultivation medium comprising a first portion of the hydrolyzate deriving from lignocellulosic biomass; and allowing the yeast to propagate to create a first fermentation broth comprising water and a first propagated yeast, wherein at least 50% of the glucose and less than 20% of the xylose in the first grow medium are consumed in the first propagation cycle. The second cycle comprises the steps of: separating the first fermentation broth into at least a first removed portion and a first residual portion, wherein both the first residual portion and the first removed portion comprise part of the first propagated yeast: contacting the first residual portion with a second cultivation medium comprising a second portion of the hydrolyzate deriving from lignocellulosic biomass; and allowing the yeast to propagate to create a second fermentation broth comprising water and a second propagated yeast, wherein at least 50% of the glucose and less than 20% of the xylose in the second grow medium are consumed in the second propagation cycle.

Hydration of the yeast in the dehydrated form used is not addressed in the above patents and patent applications.

Yeast hydration processes are known in the art.

For example, Vaudano E. et al, in “Journal of The Institute of Brewing” (2014), Vol. 120, p. 71-76, specifically report the hydration of yeasts starting from the dehydrated form. In particular, the effect of different additives on the vitality of the rehydrated yeast and its performance in fermentation is reported, using three commercial strains of Saccharomyces cerevisiae yeasts used in the production of wines. The fermentations are directly inoculated (direct pitching) and a laboratory culture medium and a grape must are used, both of which are non-toxic. The following additives are tested: inactivated dehydrated yeast (0 g/L-1 g/L), ammonium (0 mM-1.2 mM), ergosterol (0 mM-0.5 mM), ascorbic acid (0 mM-3.0 mM) and magnesium (0 mM-25 mM). Rehydration is carried out at 40° C., for 30 minutes, in a culture medium containing 5% sucrose. In particular, dehydrated yeast, which can be assimilated to yeast extract, is partially effective in a single strain, increasing the cell concentration at the end of fermentation and the ethanol produced at 48 hours of fermentation, while it has no effect on cell vitality.

Kontkanen D. et al, in “American Journal of Enology and Viticulture” (2004), Vol. 55, Issue 4, p. 363-370, report the hydration of a commercial yeast strain Saccharomyces cerevisiae used in the production of wines, starting from a non-toxic substrate. In particular, it is shown that the addition of a commercial vitamin and mineral supplement (containing pantothenate, biotin, magnesium, zinc and manganese), associated with a sufficiently high inoculum of yeast, helps to increase the growth of the same and the production of ethanol by direct inoculation during fermentation (direct pitching).

Rodríguez-Porrata B. et al, in “International Journal of Food Microbiology” (2008), Vol. 126, Issues 1-2, p. 116-122, report the possibility of improving the vitality and activity of a wine strain of Saccharomyces cerevisiae yeast through the use of additives during hydration, which takes place under the conditions suggested by the supplier (37° C., for 30 minutes, in a small volume of water). For this purpose, a variety of additives are tested (carbon and nitrogen sources, metal ions, oxidants and antioxidants, compounds that promote membrane fluidity), among which only magnesium appears to have a positive effect on the recovery of cellular activity.

Herrera T. et al, in “Archives of Biochemistry and Biophysics” (1956), Vol. 63, Issue 1, p. 131-143, describe the importance of an adequate temperature in the rehydration of Saccharomyces cerevisiae yeast used for bread making, highlighting how too low temperatures can lower the vitality of yeasts rehydrated with distilled water.

Jenkins D. M. et al, in “Journal of the Institute of Brewing” (2001), Vol. 17, Issue 3, p. 377-382, report the effect of temperature and hydration time, taking into consideration the vitality of three strains of commercial active dry yeast (ADY) used in beer fermentation. Hydration is carried out as recommended by the yeast manufacturer (non-demineralised water, for a maximum of 1 hour) at a temperature of 25° C. or 30° C.: maximum vitality is reached after 15 minutes-30 minutes of hydration and, generally, at a temperature of 30° C., despite the latter parameter being dependent on the strain in question.

Bellissimi et al, in “Process Biochemistry” (2005), Vol. 40 (6), p. 2205-2213, report the possibility of reducing the adaptation phase (lag phase) of the Saccharomyces cerevisiae yeast in fermentation in the presence of a corn hydrolyzate (first generation non-toxic sugars). In particular, the acclimatization technique is used, that is a pre-inoculation in a medium very similar to that of fermentation. The strategy makes it possible to significantly reduce the adaptation phase (lag phase) of the yeast, without however benefiting the fermentation times (which, on the contrary, are elongated). Furthermore, it is reported that the hydration of the yeast carried out in a nutrient-rich medium (peptone 10 g/L), for 20 minutes, does not show any effect on the duration of the adaptation phase (lag phase) compared to the control [direct inoculation in fermentation (direct pitching) of dehydrated yeast].

US patent application US 2004/0213889 describes a method of hydration of Saccharomyces cervevisiae yeast strains used for the production of wines or other alcoholic beverages, carried out in the presence of inactivated yeast or its derivatives. The yeast is inoculated directly during fermentation (direct pitching) of non-toxic musts and hydration is carried out using a hydration medium including yeast extract at very high concentrations (100 g/L-200 g/L) dissolved in an aqueous substrate, preferably sugary. The yeast is rehydrated in the aforementioned medium for a short period (20 minutes-40 minutes), at a temperature of 30° C.-40° C. The aforementioned method allows increasing the speed of sugar consumption in the last hours of fermentation, significantly reducing the overall time.

Most of the known art reported above relates to the production of alcoholic beverages, for which non-toxic substrates are used: therefore, there is no need to face the adaptation phase (lag phase) due to the presence of inhibitors and detoxification of the fermentation medium. Or, the adaptation phase (lag phase) is done nonetheless in the presence of hydrolyzates from first generation sugars (for example, corn hydrolyzates) that are non-toxic. Or, the problem of poor growth or the adaptation phase (lag phase) is faced and solved by modifying the yeast growth protocol, using effective but rather technologically complex methodologies (for example, use of a current of xylose only, propagation in fed-batch mode), which require particular plant set-up for their implementation on an industrial scale.

### SUMMARY

The Applicant therefore posed the problem of providing a process for hydrating yeasts in dehydrated form that allows reducing the adaptation phase (lag phase) in fermentation processes carried out in the presence of hydrolyzates deriving from lignocellulosic biomass, or of hydrolyzates containing toxic compounds, which, as mentioned above, can adversely affect fermentation.

The Applicant has now found a process, which is simple and easily achievable on an industrial scale, for hydrating a yeast in dehydrated form comprising: preparing a hydration medium comprising yeast extract in aqueous solution, at a specific concentration: adding yeast in dehydrated form to said hydration medium at a specific concentration: said process being carried out under specific conditions of temperature and time.

The aforementioned hydration process allows improving the hydration efficiency of yeasts in dehydrated form, significantly reducing the duration of the adaptation phase (lag phase) during the propagation phase, or fermentation in case of direct inoculation (direct pitching), in the presence of hydrolyzates deriving from lignocellulosic biomasses with a high content of toxic compounds (for example, acetic acid in a quantity ≥2 g/L, formic acid in a quantity ≥0.2 g/L, 5-hydroxymethylfurfural (HMF) in quantity ≥0.1 g/L, furfural (F) in a quantity ≥0.04 g/L, individually or in combination with each other). In particular, the aforementioned hydration process allows to:
reduce the overall time of yeast propagation;
reach higher cell concentrations in the fermentation broth;
reduce the risk of contamination of the yeast culture;
propagate the yeast effectively even on culture media characterized by a high content of toxic compounds without the need for dilution of the same.

The rehydrated yeast thus obtained can advantageously be used in fermentation processes that allow producing biofuels which can be used in diesel or aviation engines such as, for example, ethanol, linear or branched alcohols with different molecular weight, or biochemicals such as, for example, ethanol, glycerol, acetic acid esters (for example, ethyl acetate), arabinitol, lactic acid, succinic acid, microbial oils (bio-oils), or propagated yeast, in particular in fermentation processes carried out in the presence of hydrolyzates deriving from lignocellulosic biomass.

Therefore, the present disclosure provides a process for hydrating a yeast in dehydrated form, comprising:
preparing a hydration medium comprising yeast extract in aqueous solution, at a concentration between 1 g/L and 50 g/L, preferably between 5 g/L and 20 g/L, more preferably between 8 g/L and 15 g/L;
adding a yeast in dehydrated form to said hydration medium at a concentration between 10 g/L and 200 g/L, preferably between 12 g/L and 100 g/L, more preferably between 15 g/L and 50 g/L;
   said process being carried out at a temperature between 25° C. and 40° C., preferably between 30° C. and 35° C., for a time between 30 minutes and 8 hours, preferably between 1 hour and 7 hours, more preferably between 1.5 hours and 6.5 hours.

For the purpose of the present description and of the following claims, the definitions of the numerical ranges always include the extremes unless otherwise specified.

For the purpose of the present description and of the following claims, the term “comprising” also includes the terms “which essentially consists of” or “which consists of”.

The above process can be used for hydrating any yeast in dehydrated form subsequently used in fermentation processes that allow the production of biofuels, which can be used in diesel or aviation engines such as, for example, ethanol, linear or branched alcohols with different molecular weight, or of biochemicals such as, for example, ethanol, glycerol, esters of acetic acid (for example, ethyl acetate), arabinitol, lactic acid, succinic acid, microbial oils (bio-oils), or propagated yeast, in particular in fermentation processes carried out in the presence of hydrolyzates deriving from lignocellulosic biomass.

According to a preferred embodiment of the present disclosure, said yeast in dehydrated form can be selected, for example, from yeasts belonging to the genera: Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Rhodosporidium, Yarrowia, Schwanniomyces. Preferably, said yeast in dehydrated form is selected from yeasts belonging to the genus Saccharomyces, more preferably from strains of Saccharomyces cerevisiae, both wild and genetically modified type.

According to a preferred embodiment of the present disclosure, said aqueous solution can comprise other nutrients such as, for example, protein hydrolyzates (for example, peptone) in a quantity between 1 g/L and 40 g/L, preferably between 15 g/L and 25 g/L, sugars (for example, glucose, xylose, sucrose) in a quantity between 1 g/L and 40 g/L, preferably between 15 g/L and 25 g/L.

At the end of the hydration process, the rehydrated yeast obtained can be subjected to a propagation step, or it can be directly inoculated in fermentation (direct pitching), in the presence of hydrolyzates deriving from lignocellulosic biomass, in order to produce biofuels usable in diesel or aviation engines such as, for example, ethanol, linear or branched alcohols with different molecular weight, or biochemicals such as, for example, ethanol, glycerol, acetic acid esters (for example, ethyl acetate), arabinitol, lactic acid, succinic acid, microbial oils (bio-oils), or propagation yeast.

The present disclosure further provides a fermentation process for producing biofuels, or biochemicals, comprising the following steps:
(a) hydrating a yeast in dehydrated form to obtain a rehydrated yeast;
(b) optionally, propagating the rehydrated yeast obtained in step (a), in the presence of a culture medium comprising a hydrolyzate deriving from lignocellulosic biomass;
(c) feeding the yeast obtained in step (a) or step (b) to a fermentation device in the presence of a culture medium comprising a hydrolyzate deriving from lignocellulosic biomass;
(d) recovering, at the end of the fermentation, the biofuels or biochemicals obtained:
   characterized in that step (a) is carried out according to the process of the present disclosure.

In the above process, both in the optional step (b) (propagation) and in step (c) (fermentation), the main carbon source is the hydrolyzate deriving from lignocellulosic biomass. For this purpose, different lignocellulosic biomasses can be used: a detailed description of the lignocellulosic biomasses that can be advantageously used in the above process can be found, for example, in the international patent application WO 2015/028156, incorporated herein by reference.

According to a preferred embodiment of the present disclosure, said lignocellulosic biomass can be selected, for example, from:
plants specifically grown for energy use such as, for example, miscanthus, panicum (Panicum virgatum), common reed (Arundo donax);
plants not specifically grown for energy use such as, for example, sorghum (for example, sorghum fibers);
scraps, residues and waste of agricultural products such as, for example, guayule, corn (for example, corn stalks, corn cobs), soybeans, cotton, flax, rapeseed, wheat (for example, wheat straw), rice (for example, rice straw, rice hulls, rice husk), sugar cane (for example, sugar cane straw, sugar cane bagasse), palm (for example, palm leafs, palm trunks, palm mibrids, palm empty fruit bunches);
scraps, residues and waste of products deriving from forestation, forestry, or wood processing such as poplar, alder, birch;
scraps from agro-food products intended for human or animal husbandry consumption;
residues, not chemically treated, from the paper industry;
waste from the separate collection of municipal solid waste (for example, urban waste of vegetable origin, paper);
algae such as, for example, microalgae or macroalgae, especially macroalgae.

According to a particularly preferred embodiment of the present disclosure, said lignocellulosic biomass can be selected, for example, from: plants specifically grown for energy use such as, for example, miscanthus, panicum (Panicum virgatum), common reed (Arundo donax); plants not specifically grown for energy use such as, for example, sorghum (for example, sorghum fibers): scraps, residues and waste from agricultural products such as, for example, guayule, corn (for example, corn stalks, corn cobs), soy, cotton, flax, rapeseed, wheat (for example, wheat straw), rice (for example, rice straw, rice hulls, rice husk), sugar cane (or example, sugar cane straw, sugar cane bagasse), palm (for example, palm leafs, palm trunks, palm mibrids, palm empty fruit bunches), scraps, residues and waste of products deriving from forestation, forestry, or wood processing such as poplar, alder, birch.

Optionally, in the aforementioned steps (b) and (c), in addition to the hydrolyzate deriving from lignocellulosic biomass, other carbon sources can be added to the culture medium such as, for example, molasses, synthetic sugars: however, the monomeric sugars deriving from the hydrolysis of the lignocellulosic biomass are, preferably, at least 80% by weight, more preferably at least 90% by weight, even more preferably at least 95% by weight, of the total carbon sources used in the aforementioned steps (b) and (c).

According to a preferred embodiment of the present disclosure, said hydrolyzate deriving from lignocellulosic biomass is the only carbon source used in the aforementioned steps (b) and (c).

For the purpose of the present disclosure, the hydrolyzate deriving from lignocellulosic biomass can be obtained according to any of the hydrolysis methods known in the art.

Preferably, the hydrolyzate deriving from lignocellulosic biomass can be obtained by a process comprising the following steps: (i) a pretreatment of the starting lignocellulosic biomass to produce a pretreated lignocellulosic biomass (pretreated) in order to increase the accessibility of the polymeric and oligomeric sugars contained therein to the subsequent action of enzymes; and (ii) subjecting the pretreated lignocellulosic biomass obtained in step (i) to enzymatic hydrolysis in order to hydrolyze the polymeric and oligomeric sugars to monomeric sugars (sugars with five and six carbon atoms, mainly glucose and xylose).

Preferably, the pretreatment step (i) comprises the hydrothermal treatment of the starting lignocellulosic biomass with water, in a vapor phase, in a pressurized reactor, and the subsequent steam explosion of the lignocellulosic biomass after hydrothermal treatment carried out by quickly releasing the pressure applied to the lignocellulosic biomass.

Preferably, before the pretreatment step (i), the starting lignocellulosic biomass can be subjected to a soaking step (i′) in order to remove a part of non-lignocellulosic compounds contained in the starting lignocellulosic biomass such as, for example, inorganic salts, waxes, organic acids. During said step (i′) of soaking with water, external contaminants such as, for example, dust, stones, collection residues can also be removed.

More details relating to said pretreatment step (i) and to said soaking step (i′), can be found, for example, in european patent EP 3,241,907, incorporated herein by reference.

Optionally, said pretreatment step (i) can be carried out in two steps as described, for example, in european patent EP 2,414,531, incorporated herein by reference.

The above step (ii) of enzymatic hydrolysis can be carried out by putting in contact an aqueous suspension (slurry) of lignocellulosic biomass obtained in step (i), with an enzyme or a mixture (cocktail) of enzymes, operating under conditions able to promote said enzymatic hydrolysis.

In order to obtain said aqueous suspension (slurry), water, possibly recirculating water, can be added to the pretreated lignocellulosic biomass obtained in step (i), or an aqueous solution containing sugars (for example, an aqueous solution containing sugars from enzymatic hydrolysis), in a quantity such as to obtain a suspension (slurry) comprising a quantity of pretreated biomass (dry weight) preferably between 10% by weight and 25% by weight, with respect to the total weight of said aqueous suspension (slurry), can be added.

Preferably, the above step (ii) of enzymatic hydrolysis can be carried out under stirring at a pH between 4.5 and 5.3 at a temperature between 45° C. and 65° C., and for a time between 24 hours and 120 hours.

The above step (ii) of enzymatic hydrolysis can comprise one or more steps.

At the end of said hydrolysis step (ii), a mixture is obtained comprising a solid residue (i.e. solid phase) comprising pretreated non-solubilized lignocellulosic biomass (i.e. insoluble solids, mainly lignin) and a hydrolyzate from lignocellulosic biomass (i.e. aqueous phase) comprising water and monomeric sugars with five and six carbon atoms, mainly glucose and xylose.

Optionally, all or at least a part of said solid residue (i.e. solid phase) comprising pretreated non-solubilized lignocellulosic biomass (i.e. insoluble solids, mainly lignin), can be removed from the above mixture according to techniques known in the art such as, for example, decanting, centrifugation, or pressing, or a combination thereof.

In a preferred embodiment of the present disclosure, no removal of said solid residue (i.e. solid phase) is carried out comprising pretreated non-solubilized lignocellulosic biomass (i.e. insoluble solids, mainly lignin) and, consequently, the hydrolyzate deriving from lignocellulosic biomass used in steps (b) and (c) of the above process, is the above mixture comprising a solid residue (ie solid phase) comprising pretreated non-solubilized lignocellulosic biomass (i.e. insoluble solids, mainly lignin) and a hydrolyzate from lignocellulosic biomass (i.e. phase aqueous) comprising water and monomeric sugars with five and six carbon atoms, mainly glucose and xylose.

Preferably, for the purpose of the present disclosure, the enzymatic hydrolysis of the lignocellulosic biomass can be carried out by operating as described, for example, in the international patent application WO 2010/113130, incorporated herein by reference.

The enzymatic hydrolysis processes reported above are to be considered exemplary and preferred embodiments for the purpose of obtaining the hydrolyzate deriving from lignocellulosic biomass and not limiting the present disclosure.

According to a preferred embodiment of the present disclosure, said hydrolyzate deriving from lignocellulosic biomass is a mixture comprising a solid residue (i.e. solid phase) comprising pretreated non-solubilized lignocellulosic biomass (i.e. insoluble solids, mainly lignin) and a hydrolyzate from lignocellulosic biomass (i.e. aqueous phase) comprising water and monomeric sugars with five and six carbon atoms, mainly glucose and xylose, said mixture having a total solids content (soluble+insoluble) between 5% by weight and 30% by weight, preferably between 10% by weight and 20% by weight, with respect to the total weight of said mixture.

According to a preferred embodiment of the present disclosure, said hydrolyzate deriving from lignocellulosic biomass is a mixture comprising a solid residue (i.e. solid phase) comprising pretreated non-solubilized lignocellulosic biomass (i.e. insoluble solids, mainly lignin) and a hydrolyzate from lignocellulosic biomass (i.e. aqueous phase) comprising water and monomeric sugars with five and six carbon atoms, mainly glucose and xylose, said aqueous phase having a glucose content between 20 g/L and 100 g/L, preferably between 30 g/L and 70 g/L, more preferably between 40 g/L and 60 g/L.

According to a preferred embodiment of the present disclosure, said hydrolyzate deriving from lignocellulosic biomass is a mixture comprising a solid residue (i.e. solid phase) comprising unsolubilized pre-treated lignocellulosic biomass (i.e. insoluble solids, mainly lignin) and a hydrolyzate from lignocellulosic biomass (i.e. aqueous phase) comprising water and monomeric sugars with five and six carbon atoms, mainly glucose and xylose, said aqueous phase having a xylose content between 10 g/L and 40 g/L, preferably between 15 g/L and 30 g/L, more preferably between 18 g/L and 25 g/L.

Said hydrolyzate deriving from lignocellulosic biomass also includes toxic compounds such as, for example, acetic acid, formic acid, 5-hydroxy methylfurfural (HMF), furfural (F) which, as mentioned above, can act as growth inhibitors of the microorganisms usually used in fermentation. According to a preferred embodiment of the present disclosure, said hydrolyzate deriving from lignocellulosic biomass is a mixture comprising a solid residue (i.e. solid phase) comprising pretreated non-solubilized lignocellulosic biomass (i.e. insoluble solids, mainly lignin) and a hydrolyzate from lignocellulosic biomass (i.e. aqueous phase) comprising water and monomeric sugars with five and six carbon atoms, mainly glucose and xylose, said aqueous phase having an acetic acid content between 2 g/L and 8 g/L, preferably between 2.5 g/L and 7.5 g/L; and/or formic acid between 0.2 g/L and 2 g/L, preferably between 0.3 g/L and 1.8 g/L; and/or 5-hydroxymethylfurfural (HMF) between 0.1 g/L and 1 g/L, preferably between 0.2 g/L and 0.8 g/L; and/or furfural (F) between 0.04 g/L and 1 g/L, preferably between 0.05 g/L and 0.8 g/L.

According to a further embodiment of the present disclosure, said hydrolyzate deriving from lignocellulosic biomass is the aqueous phase comprising water and monomeric sugars with five and six carbon atoms, mainly glucose and xylose deriving from the hydrolysis of the lignocellulosic biomass, said aqueous phase having a glucose content between 20 g/L and 100 g/L, preferably between 30 g/L and 70 g/L, more preferably between 40 g/L to 60 g/L.

According to a further embodiment of the present disclosure, said hydrolyzate deriving from lignocellulosic biomass is the aqueous phase comprising water and monomeric sugars with five and six carbon atoms, mainly glucose and xylose deriving from the hydrolysis of the lignocellulosic biomass, said aqueous phase having a xylose content between 10 g/L and 40 g/L, preferably between 15 g/L and 30 g/L, more preferably between 18 g/L and 25 g/L.

According to a further embodiment of the present disclosure, said hydrolyzate deriving from lignocellulosic biomass is the aqueous phase comprising water and monomeric sugars with five and six carbon atoms, mainly glucose and xylose deriving from the hydrolysis of the lignocellulosic biomass, said aqueous phase having an acetic acid content between 2 g/L and 8 g/L, preferably between 2.5 g/L and 7.5 g/L; and/or formic acid between 0.2 g/L and 2 g/L, preferably between 0.3 g/L and 1.8 g/L; and/or 5-hydroxymethylfurfural (HMF) between 0.1 g/L and 1 g/L, preferably between 0.2 g/L and 0.8 g/L; and/or furfural (F) between 0.04 g/L and 1 g/L, preferably between 0.05 g/L and 0.8 g/L.

Furthermore, said hydrolyzate deriving from lignocellulosic biomass can contain biological contaminants, as typically occurs in processes on an industrial scale, generally carried out in non-sterile environments. Said biological contaminants are microbial organisms other than yeast which one wishes to propagate and/or subject to fermentation according to the process of the present disclosure, and whose presence generally negatively affects its yield, as part of the monomeric sugars are consumed by said microbial organisms and are no longer available for the growth of the desired yeast. Said biological contaminants can include bacteria and/or fungi, as well as yeasts other than the desired yeast.

In order to avoid contamination due to said biological contaminants in propagation and/or fermentation, said hydrolyzate deriving from lignocellulosic biomass, the means of propagation and/or fermentation and all further equipment involved in the process, can be subjected to sterilization by methods known in the art such as, for example, the addition of one or more antibacterial agents (for example, antibiotics or other aseptic agents), sterilization processes (for example, pasteurization), application of physical agents (for example, heat, light, radiation), before, or during the propagation and/or fermentation phases. By sterilization we mean here the reduction of the density of biological contaminants by a factor of at least 100.

However, it should be noted that, preferably, for the purpose of the process of the present disclosure, the use of antibacterial agents and sterilization processes, which introduces additional costs and is difficult to apply at an industrial level, is avoided.

According to a preferred embodiment of the present disclosure said step (b) can be carried out at a temperature between 20° C. and 45° C., preferably between 25° C. and 35° C.

According to a preferred embodiment of the present disclosure, said step (b) can be carried out for a time ranging from 4 hours to 30 hours, preferably from 8 hours to 24 hours.

According to a preferred embodiment of the present disclosure, said step (b) can be carried out at a pH between 4 and 8, preferably between 4.5 and 7. In order to maintain the pH in the desired ranges, an aqueous solution of at least one inorganic base such as, for example, sodium hydroxide, potassium hydroxide, calcium hydroxide, magnesium hydroxide, or mixtures thereof, preferably sodium hydroxide: or an aqueous solution of at least one inorganic acid such as, for example, phosphoric acid, sulfuric acid, 2-(N-morpholino) ethane sulfonic acid (MES), hydrochloric acid, or mixtures thereof; in such quantity as to obtain the desired pH, can be added to the culture medium used in said step (b). Preferably, an aqueous solution of sulfuric acid can be added.

According to a preferred embodiment of the present disclosure, said step (b) can be carried out at an air flow rate between 1 vvh and 120 vvh, preferably between 5 vvh and 30 vvh (volume of air flowing per volume of culture medium).

According to a preferred embodiment of the present disclosure, in said step (b), in addition to the hydrolyzate deriving from lignocellulosic biomass, various nutrients such as, for example, nitrogen sources (for example, urea), potassium phosphate, magnesium, salts, vitamins, microelements, can be added to the culture medium.

According to a preferred embodiment of the present disclosure, said step (c) can be carried out at a temperature between 20° C. and 45° C., preferably between 25° C. and 35° C.

According to a preferred embodiment of the present disclosure, said step (c) can be carried out for a time between 24 hours and 140 hours, preferably between 40 hours and 120 hours.

According to a preferred embodiment of the present disclosure, said step (c) can be carried out at a pH between 4 and 8, preferably between 4.5 and 7. In order to maintain the pH in the desired ranges, an aqueous solution of at least one inorganic base such as, for example, sodium hydroxide, potassium hydroxide, calcium hydroxide, magnesium hydroxide, or mixtures thereof, preferably sodium hydroxide: or an aqueous solution of at least one inorganic acid such as, for example, phosphoric acid, sulfuric acid, 2-(N-morpholino) ethane sulfonic acid (MES), hydrochloric acid, or mixtures thereof; in such quantity as to obtain the desired pH, can be added to the culture medium used in said step (c). Preferably, an aqueous solution of sulfuric acid can be added.

According to a preferred embodiment of the present disclosure, said step (c) can be carried out in the absence of air.

According to a preferred embodiment of the present disclosure, said step (c) can be carried out at an air flow rate between 1 vvh and 120 vvh, preferably between 10 vvh and 60 vvh (volume of air flowing per volume of culture medium for now).

According to a preferred embodiment of the present disclosure, in said step (c), in addition to the hydrolyzate deriving from lignocellulosic biomass, various nutrients such as, for example, nitrogen sources (for example, urea), potassium phosphate, magnesium, salts, vitamins, microelements, can be added to the culture medium.

According to a preferred embodiment of the present disclosure, said step (c) is a fermentation in batch, or in a discontinuous culture (fed-batch fermentation), or in a continuous culture, preferably in batch.

Said step (d) can be carried out according to processes known in the art such as, for example, distillation, centrifugation, extraction.

### DETAILED DESCRIPTION OF THE DISCLOSURE

In order to better understand the present disclosure and to put it into practice, some illustrative and non-limiting examples thereof are reported below.

### EXAMPLE 1

Analysis of the Hydrolyzate Deriving from Lignocellulosic Biomass

For this purpose, a mixture deriving from the enzymatic hydrolysis of white poplar veneer was used as hydrolyzate comprising an aqueous phase comprising water and monomeric sugars with five and six carbon atoms and a solid phase comprising insoluble solids in an amount equal to 6.5% by weight with respect to the total weight of said mixture.

The content of insoluble solids was determined in accordance with the NREL standard reported in the Technical Report NREL/TP-510-42627: “Determination of Insoluble Solids in Pretreated Biomass Material”, Laboratory Analytical Procedure (LAP) (Issue Date: Mar. 21, 2008) available at the following website: https://www.nrel.gov/docs/gen/fy08/42627.pdf.

The total solids content (soluble+insoluble) was determined in accordance with the NREL standard reported in the Technical Report NREL/TP-510-42621 “Determination of Total Solids in Biomass and Total Dissolved Solids in Liquid Process Samples”, Laboratory Analytical Procedure (LAP) (Issue Date: Mar. 31, 2008) available at the following website: https://www.nrel.gov/docs/gen/fy08/42621.pdf.

The composition of said aqueous phase was determined in accordance with the NREL standard reported in the Technical Report NREL/TP-510-42623“Determination of Sugars, Byproducts, and Degradation Products in Liquid Fraction Process Samples”, Laboratory Analytical Procedure (LAP) (Issue Date: Dec. 8, 2006) available at the following website: https://www.nrel.gov/docs/gen/fy08/42623.pdf.

TABLE 1
WATER PHASE COMPOSITION

| | | |
|---|---|---|
| Glucose | g/L | 55.1 |
| Xylose | g/L | 19.1 |
| Arabinose | g/L | 0.19 |
| Formic acid | g/L | 0.8 |
| Lactic acid | g/L | 0.13 |
| Acetic acid | g/L | 5.3 |
| 5-hydroxymethylfurfural | g/L | 0.25 |
| (HMF) | | |
| Furfural (F) | g/L | 0.06 |
| Glucoligomers | g/L | 3.84 |
| Xyloligomers | g/L | 3.84 |

INSOLUBLE SOLIDS CONTENTS IN THE SOLID PHASE

| | | |
|---|---|---|
| Insoluble solids | % by weight | 6.5 |

TOTAL SOLID CONTENT IN THE SOLID PHASE

| | | |
|---|---|---|
| Total solids (soluble + | % by weight | 14.7 |
| insoluble) | | |

As mentioned above, acetic acid, formic acid, 5-hydroxymethylfurfural (HMF), furfural (F), are toxic compounds that act as growth inhibitors of the yeast Saccharomyces cerevisiae used in the following examples of propagation while, the lactic acid is produced by bacterial contamination. However, no antibiotics were added.

### EXAMPLE 2 (COMPARATIVE)

Propagation of Yeast Saccharomyces cerevisiae on Hydrolyzate Deriving from Lignocellulosic Biomass with Nutrient-Free Hydration

The commercial yeast strain Saccharomyces cerevisiae (Ethanol Red® yeast—Leaf by Lesaffre), preserved in dehydrated form, was rehydrated according to the manufacturer's instructions: 30 minutes in physiological solution (NaCl 0.9% by weight, in a quantity equal to 5 times the weight of the yeast) at a temperature of 32° C., under stirring (100 rpm), in a 50 ml test tube.

Subsequently, the yeast was inoculated in a 1-liter bioreactor, at a concentration of 0.25 g/L, in the hydrolyzate deriving from lignocellulosic biomass obtained from white poplar veneer described in Example 1 previously centrifuged at 6000 rpm, for 15 minutes, in order to eliminate the solid phase. Urea at a concentration of 1.5 g/L was added to the hydrolyzate deriving from lignocellulosic biomass.

The propagation was carried out at 32° C., pH 5.3, aeration 10 vvh, in batch mode, in a 1 L reactor with a maximum volume at 66% filling, at a fixed stirring of 500 rpm.

Cell budding was observed 4 hours after inoculation, while the increase in cell concentration (cell growth) it started at 6 hours. After 18 hours of propagation, a cell concentration of 1.5 g/L (6× multiplication factor) was measured.

Cell budding was observed using a Burker chamber microscope.

Cell growth during fermentation was measured by counting under a Burker chamber microscope and by determining the dry weight of the cell biomass in an oven, at 105° C.

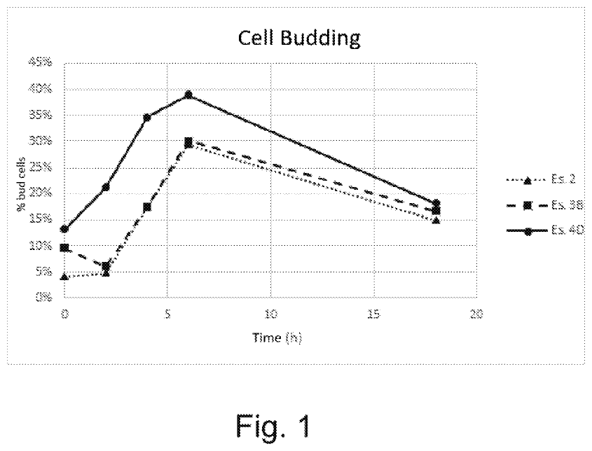

FIG. 1 shows the cell budding curve (the % of bud cells is shown on the ordinate: the time in hours on the abscissa).

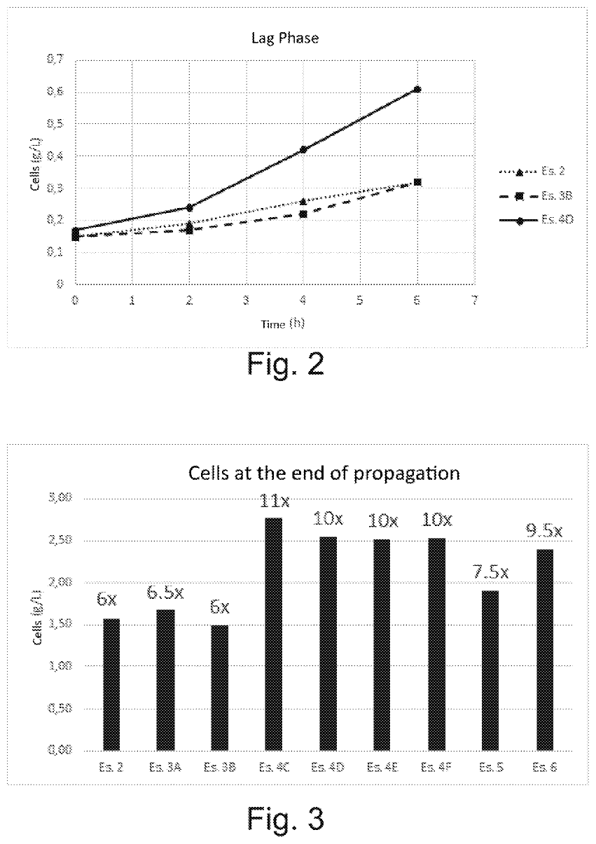

FIG. 2 shows the curve of the adaptation phase (lag phase) (the concentration of cells expressed in g/L is shown on the ordinate; the time in hours on the abscissa).

FIG. 3 shows the cell concentration at the end of propagation (the concentration of cells expressed in g/L is shown on the ordinate: the number of the example and, where necessary, the letter that identifies the sample on the abscissa).

### EXAMPLE 3 (COMPARATIVE)

Propagation of Yeast Saccharomyces cerevisiae on Hydrolyzate Deriving from Lignocellulosic Biomass, with Different Hydration Times, without Nutrients

The commercial yeast strain Saccharomyces cerevisiae (Ethanol Red® yeast—Leaf by Lesaffre), preserved in dehydrated form, was rehydrated, for 6 hours (Sample A), and for 2 hours (Sample B), in physiological solution (NaCl 0.9% by weight) at a concentration of 16 g/L (weight of the yeast with respect to the volume of hydration), at a temperature of 32° C., under stirring (200 rpm), in a 100 ml flask.

Subsequently, the yeast was inoculated in a 1-liter bioreactor, at a concentration of 0.25 g/L, in the hydrolyzate deriving from lignocellulosic biomass obtained from white poplar veneer described in Example 1 previously centrifuged at 6000 rpm, for 15 minutes, in order to eliminate the solid phase. Urea at a concentration of 1.5 g/L was added to the hydrolyzate deriving from lignocellulosic biomass.

The propagation was carried out at 32° C., pH 5.3, aeration 10 vvh, in batch mode, in a 1 L reactor with a maximum volume at 66% filling, at a fixed stirring of 500 rpm.

In Sample B (hydration of 2 hours), cell budding was observed 4 hours after inoculation, while the increase in cell concentration (cell growth) started at 6 hours.

After 18 hours of propagation, a cell concentration equal to:
Sample A: 1.7 g/L (6× multiplication factor);
Sample B: 1.5 g/L (6.5× multiplication factor).

FIG. 1 shows the cell budding curve for Sample B (Ex. 2B) (the % of bud cells is shown on the ordinate; the time in hours on the abscissa).

FIG. 2 shows the curve of the adaptation phase (lag phase) for Sample B (Ex. 3B) (the concentration of cells expressed in g/L is shown on the ordinate; the time in hours on the abscissa).

FIG. 3 shows the cell concentration at the end of propagation for Sample A (Ex. 3A) and for Sample B (Ex. 3B) (the concentration of cells expressed in g/L is shown on the ordinate: the number of the example and, where necessary, the letter that identifies the sample on the abscissa).

### EXAMPLE 4 (DISCLOSURE)

Propagation of Yeast Saccharomyces cerevisiae on Hydrolyzate Deriving from Lignocellulosic Biomass, with Hydration in the Presence of Nutrients

The commercial yeast strain Saccharomyces cerevisiae (Ethanol Red® yeast—Leaf by Lesaffre), preserved in dehydrated form, was rehydrated, in different aqueous solutions and at different times as reported below, at a concentration of 16 g/L (weight of the yeast with respect to the volume of hydration), at a temperature of 32° C., under stirring (200 rpm), in a 100 ml flask.

The following aqueous solutions and times were used:
Sample C: yeast extract 10 g/L+peptone 20 g/L+glucose 20 g/L: 6 hours;
Sample D: yeast extract 10 g/L+peptone 20 g/L+glucose 20 g/L: 2 hours;
Sample E: yeast extract 10 g/L: 6 hours;
Sample F: yeast extract 10 g/L: 2 hours.

Subsequently, the yeast was inoculated in a 1-liter bioreactor, at a concentration of 0.25 g/L, in the hydrolyzate deriving from lignocellulosic biomass obtained from white poplar veneer described in Example 1 previously centrifuged at 6000 rpm, for 15 minutes, in order to eliminate the solid phase. Urea at a concentration of 1.5 g/L was added to the hydrolyzate deriving from lignocellulosic biomass.

The propagation was carried out at 32° C., pH 5.3, aeration 10 vvh, in batch mode, in a 1-L reactor with a maximum volume at 66% filling, at a fixed stirring of 500 rpm.

In all samples, cell budding was already evident at the time of inoculation, while the increase in cell concentration (cell growth) started at 2 hours.

After 18 hours of propagation, a cell concentration equal to:
Sample C: 2.7 g/L (11× multiplication factor);
Sample D: 2.5 g/L (10× multiplication factor);
Sample E: 2, 5 g/L (10× multiplication factor);
Sample F: 2.5 g/L (10× multiplication factor).

FIG. 1 shows the cell budding curve for Sample D (Ex. 4D) (the % of bud cells is shown on the ordinate; the time in hours on the abscissa).

FIG. 2 shows the curve of the adaptation phase (lag phase) for Sample D (Ex. 4D) (the concentration of cells expressed in g/L is shown on the ordinate; the time in hours on the abscissa).

FIG. 3 shows the cell concentration at the end of propagation for Sample C (Ex. 4C), for Sample D (Ex. 4D), for Sample E (Ex. 4E), for Sample F (Ex. 4F) (the concentration of cells expressed in g/L is shown on the ordinate; the number of the example and, where necessary, the letter that identifies the sample on the abscissa).

### EXAMPLE 5 (COMPARATIVE)

Propagation of Yeast Saccharomyces cerevisiae on Hydrolyzate Deriving from Lignocellulosic Biomass, with Nutrient-Free Hydration and Propagation in the Presence of Nutrients

commercial yeast strain Saccharomyces cerevisiae (Ethanol Red & yeast-Leaf by Lesaffre), preserved in dehydrated form, was rehydrated according to the manufacturer's instructions: 30 minutes in physiological solution (NaCl 0.9% by weight, in an equal quantity 5 times the weight of the yeast), at a temperature of 32° C., under stirring (100 rpm), in a 50 ml test tube.

Subsequently, the yeast was inoculated in a 1-liter bioreactor, at a concentration of 0.25 g/L, in the hydrolyzate deriving from lignocellulosic biomass obtained from white poplar veneer described in Example 1 previously centrifuged at 6000 rpm, for 15 minutes, in order to eliminate the solid phase. Urea at a concentration of 1.5 g/L and yeast extract in the same quantity that would result from the hydration described in Example 3 above (resulting concentration 0.15 g/L), was added to the hydrolyzate deriving from lignocellulosic biomass.

The propagation was carried out at 32° C., pH 5.3, aeration 10 vvh, in batch mode, in a 1 L reactor with a maximum volume at 66% filling, at a fixed stirring of 500 rpm.

Cell budding was observed 4 hours after inoculation, while the increase in cell concentration (cell growth) started at 6 hours. After 18 hours of propagation, a cell concentration of 1.9 g/L (7.5× multiplication factor) was measured.

FIG. 3 shows the cell concentration at the end of propagation (the concentration of cells expressed in g/L is shown on the ordinate; the number of the example and, where necessary, the letter that identifies the sample on the abscissa).

### EXAMPLE 6 (DISCLOSURE)

Propagation of Yeast Saccharomyces cerevisiae on Hydrolyzate Deriving from Lignocellulosic Biomass, with Hydration in the Presence of Nutrients

The commercial yeast strain Saccharomyces cerevisiae (Ethanol Red® yeast—Leaf by LeSaffre), preserved in dehydrated form, was rehydrated in an aqueous solution comprising yeast extract at a concentration of 10 g/L, for 2 hours, at a concentration of 16 g/L (weight of the yeast with respect to the volume of hydration), at a temperature of 32° C., under gentle stirring (200 rpm), in a 100 ml flask.

Subsequently, the yeast was inoculated in a 1-liter bioreactor, at a concentration of 0.25 g/L, in the hydrolyzate deriving from lignocellulosic biomass obtained from white poplar veneer described in Example 1, not subjected to the removal of the solid phase. Urea at a concentration of 1.5 g/L and yeast extract in the same quantity that would result from the hydration described in Example 3 above (resulting concentration 0.15 g/L), was added to the hydrolyzate deriving from lignocellulosic biomass.

The propagation was carried out at 32° C., pH 5.3, aeration 10 vvh, in batch mode, in a 1 L reactor with a maximum volume at 66% filling, at a fixed stirring of 500 rpm.

Cell budding was observed 4 hours after inoculation, while the increase in cell concentration (cell growth) started at 6 hours. After 24 hours of propagation, a cell concentration of 2.4 g/L (9.5× multiplication) was measured.

Since it was not possible to directly measure the dry cell weight due to the presence of insoluble solids in the lignocellulosic hydrolyzate, the cell concentration was measured by counting the cells under a Burker chamber microscope. The relative dry cell weight was calculated by means of a calibration line, which correlates number of cells and dry weight, built in an identical culture medium deprived of insoluble solids.

FIG. 3 shows the cell concentration at the end of propagation (the concentration of cells expressed in g/L is shown on the ordinate; the number of the example and, where necessary, the letter that identifies the sample on the abscissa).

1. A process for hydrating yeasts in dehydrated form, the process including the following steps:
preparing a hydration medium comprising yeast extract in aqueous solution, at a concentration between 1 g/L and 50 g/L; and
adding a yeast in dehydrated form to said hydration medium at a concentration between 10 g/L and 200 g/L;
said process being carried out at a temperature between 25° C. and 40° C., for a time between 30 minutes and 8 hours.

2. The process for hydrating yeasts in dehydrated form according to claim 1, wherein said yeast in dehydrated form is selected from yeasts belonging to the genera: Saccharomyces, Zygosaccharomyces, Candida, Hansemila, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Rhodosporidium, Yarrowia, Schwanniomyces.

3. The process for hydrating yeasts in dehydrated form according to claim 1, wherein said aqueous solution comprises other nutrients such as protein hydrolyzates (such as peptone) in a quantity between 1 g/L and 40 g/L, sugars (such as glucose, xylose, sucrose) in a quantity between 1 g/L and 40 g/L.

4. A fermentation process for producing biofuels or biochemicals, the process including the following steps:
(a) hydrating a yeast in a dehydrated form to obtain a rehydrated yeast;
(b) optionally, propagating the rehydrated yeast obtained in step (a), in the presence of a culture medium comprising a hydrolyzate deriving from lignocellulosic biomass;
(c) feeding the yeast obtained in step (a) or step (b) to a fermentation device in the presence of a culture medium comprising a hydrolyzate deriving from lignocellulosic biomass;
(d) recovering, at the end of the fermentation, the biofuels or biochemicals obtained;
whereby step (a) is carried out according to the process of claim 1.

5. The fermentation process for producing biofuels or biochemicals according to claim 4, wherein said lignocellulosic biomass is selected from:
plants specifically grown for energy use such as miscanthus, panicum (Panicum virgatum), common reed (Arundo donax);
plants not specifically grown for energy use such as sorghum (such as sorghum fibers);
scraps, residues and waste of agricultural products such as guayule, corn (such as corn stalks, corn cobs), soybeans, cotton, flax, rapeseed, wheat (such as wheat straw), rice (such as rice straw, rice hulls, rice husk), sugar cane (such as sugar cane straw, sugar cane bagasse), palm (such as palm leafs, palm trunks, palm mibrids, palm empty fruit bunches);
scraps, residues and waste of products deriving from forestation, forestry, or wood processing such as poplar, alder, birch;
scraps from agro-food products intended for human or animal husbandry consumption;
residues, not chemically treated, from the paper industry;
waste from the separate collection of municipal solid waste (such as urban waste of vegetable origin, paper); and
algae such as microalgae or macroalgae, especially macroalgae.

6. The fermentation process for producing biofuels or biochemicals according to claim 5, wherein said lignocellulosic biomass is selected from: plants specifically grown for energy use such as miscanthus, panicum (Panicum virgatum), common reed (Arundo donax); plants not specifically grown for energy use such as sorghum (such as sorghum fibers); scraps, residues and waste from agricultural products such as guayule, corn (such as corn stalks, corn cobs), soy, cotton, flax, rapeseed, wheat (such as wheat straw), rice (such as rice straw, rice hulls, rice husk), sugar cane (such as sugar cane straw, sugar cane bagasse), palm (such as palm leafs, palm trunks, palm mibrids, palm empty fruit bunches), scraps, residues and waste of products deriving from forestation, forestry, or wood processing such as poplar, alder, birch.

7. The fermentation process for producing biofuels or biochemicals according to claim 4, wherein said hydrolyzate deriving from lignocellulosic biomass is the only carbon source used in the aforementioned steps (b) and (c).

8. The fermentation process for producing biofuels or biochemicals according to claim 4, wherein said hydrolyzate deriving from lignocellulosic biomass is a mixture comprising a solid residue (i.e. solid phase) comprising pretreated non-solubilized lignocellulosic biomass (i.e. solid insoluble, mainly lignin) and a hydrolyzate from lignocellulosic biomass (i.e. aqueous phase) comprising water and monomeric sugars with five and six carbon atoms, mainly glucose and xylose, said mixture having a content of total solids (soluble+insoluble) between 5% by weight and 30% by weight, with respect to the total weight of said mixture.

9. The fermentation process for producing biofuels or biochemicals according to claim 4, wherein said hydrolyzate deriving from lignocellulosic biomass is a mixture comprising a solid residue (i.e. solid phase) comprising pretreated non-solubilized lignocellulosic biomass (i.e. solid insoluble, mainly lignin) and a hydrolyzate from lignocellulosic biomass (i.e. aqueous phase) comprising water and monomeric sugars with five and six carbon atoms, mainly glucose and xylose, said aqueous phase having a glucose content between 20 g/L and 100 g/L.

10. The fermentation process for producing biofuels or biochemicals according to claim 4, wherein said hydrolyzate deriving from lignocellulosic biomass is a mixture comprising a solid residue (i.e. solid phase) comprising pretreated non-solubilized lignocellulosic biomass (i.e. solid insoluble, mainly lignin) and a hydrolyzate from lignocellulosic biomass (i.e. aqueous phase) comprising water and monomeric sugars with five and six carbon atoms, mainly glucose and xylose, said aqueous phase having a xylose content between 10 g/L and 40 g/L.

11. The fermentation process for producing biofuels or biochemicals according to claim 4, wherein said hydrolyzate deriving from lignocellulosic biomass is a mixture comprising a solid residue (i.e. solid phase) comprising pretreated non-solubilized lignocellulosic biomass (i.e. solids insoluble, mainly lignin) and a hydrolyzate from lignocellulosic biomass (i.e. aqueous phase) comprising water and monomeric sugars with five and six carbon atoms, mainly glucose and xylose, said aqueous phase having an acetic acid content between 2 g/L and 8 g/L; and/or formic acid between 0.2 g/L and 2 g/L; and/or 5-hydroxymethylfurfural (HMF) between 0.1 g/L and 1 g/L; and/or furfural (F) between 0.04 g/L and 1 g/L.

12. The fermentation process for producing biofuels or biochemicals according to claim 4, wherein said hydrolyzate deriving from lignocellulosic biomass is the aqueous phase comprising water and monomeric sugars with five and six carbon atoms, mainly glucose and xylose deriving from the hydrolysis of the lignocellulosic biomass, said aqueous phase having a glucose content between 20 g/L and 100 g/L.

13. The fermentation process for producing biofuels or biochemicals according to claim 4, wherein said hydrolyzate deriving from lignocellulosic biomass is the aqueous phase comprising water and monomeric sugars with five and six carbon atoms, mainly glucose and xylose deriving from the hydrolysis of the lignocellulosic biomass, said aqueous phase having a xylose content between 10 g/L and 40 g/L.

14. The fermentation process for producing biofuels or biochemicals according to claim 4, wherein said hydrolyzate deriving from lignocellulosic biomass is the aqueous phase comprising water and monomeric sugars with five and six carbon atoms, mainly glucose and xylose deriving from the hydrolysis of the lignocellulosic biomass, said aqueous phase having an acetic acid content between 2 g/L and 8 g/L; and/or formic acid between 0.2 g/L and 2 g/L; and/or 5-hydroxymethylfurfural (HMF) between 0.1 g/L and 1 g/L; and/or furfural (F) between 0.04 g/L and 1 g/L.

15. The fermentation process for producing biofuels or biochemicals according to claim 4, wherein said step (b) is carried out:
at a temperature between 20° C. and 45° C.; and/or
for a time between 4 hours and 30 hours; and/or
at a pH between 4 and 8; and/or
at an air flow rate between 1 vvh and 120 vvh, (volume of air flowing per volume of culture medium); and/or
in a culture medium wherein, in addition to the hydrolyzate deriving from lignocellulosic biomass, various nutrients are added such as nitrogen sources (such as urea), potassium phosphate, magnesium, salts, vitamins, microelements.

16. The fermentation process for producing biofuels or biochemicals according to claim 4, wherein said step (c) is carried out:
at a temperature between 20° C. and 45° C.; and/or
for a time between 24 hours and 140 hours; and/or
at a pH between 4 and 8; and/or
in the absence of air; or at an air flow rate between 1 vvh and 120 vvh, (volume of air flowing per volume of culture medium per hour); and/or
in a culture medium wherein, in addition to the hydrolyzate deriving from lignocellulosic biomass, various nutrients are added such as nitrogen sources (such as urea), potassium phosphate, magnesium, salts, vitamins, microelements; and/or
using fermentation in batch, or discontinuous culture (fed-batch fermentation), or continuous culture.
